SEQUENCE LISTING (3 sequences)

SEQ ID NO: 1 - protein, 9 aa, Artificial sequence
  Synthesised peptide
CGPLGLLAR

SEQ ID NO: 2 - protein, 5 aa, Artificial sequence
  Syntehsised peptide
DFKRG

SEQ ID NO: 3 - protein, 5 aa, Artificial sequence
  Synthesised peptide
  MISC_FEATURE (1)..(5): Cyclisation of peptide through peptide bond between amino acid 1 and 5
DFKRG

(19) United States
(12) Patent Application Publication
(10) Pub. No.: US 2006/0292078 A1
(43) Pub. Date: Dec. 28, 2006

(54) Optical imaging of colorectal cancer

(76) Inventors: Jo Klaveness, Oslo (NO); Edwin Johannesen, Oslo (NO); Helge Tolleshaug, Oslo (NO)

Correspondence Address: GE HEALTHCARE, INC., IP DEPARTMENT, 101 CARNEGIE CENTER, PRINCETON, NJ, 08540-6231, US

(21) Appl. No.: 10/573,606

(22) PCT Filed: Sep. 28, 2004
(86) PCT No.: PCT/NO04/00287

§ 371 Date: Mar. 28, 2006

(30) Foreign Application Priority Data
Sep. 29, 2003 (NO) 20034350

(51) Int. Cl. A61K 49/00; C07K 7/06; C07K 7/08

(52) U.S. Cl. 424/9.6; 530/329; 530/330

(57) ABSTRACT

The invention provides contrast agents for optical imaging of colorectal cancer (CRC) in patients. The contrast agents may be used in diagnosis of CRC, for follow up of progress in disease development, and for follow up of treatment of CRC. Further, the invention provides methods for optical imaging of CRC in patients.

### FIELD OF THE INVENTION

The present invention provides contrast agents for optical imaging of colorectal cancer (CRC) in patients. The contrast agents may be used in diagnosis of CRC, for follow up of progress in disease development, and for follow up of treatment of CRC.

The present invention also provides new methods of optical imaging of CRC in patients, for diagnosis and for follow up of disease development and treatment of CRC.

### DESCRIPTION OF RELATED ART

Colorectal cancer (CRC) is one of the most frequent malignant diseases in the Western civilization. More than 100 000 new cases of CRC are diagnosed every year in US and the disease is fatal for a high percentage of these. CRC is the fourth most commonly diagnosed cancer disease and it is the second leading cause of cancer death in US after lung cancer.

The peak incidence of CRC generally occurs after the age of 60 years. CRC is more common in the western world than, in underdeveloped countries. There may be several reasons for this, including life expectancy, genetic susceptibility and diet. It has been suggested that, intake of fat and red meat has a negative effect on the incidence of CRC while a fibre rich diet might decrease the risk for CRC.

Most of the CRC incidents are adenocarcinomas. The sizes of the lesions are normally in the range from a few millimetres to several centimetres, and the lesions are unevenly distributed througouth the lower part of the gastrointestinal system. Normally CRC cells remain superficial for a long time and will slowly invade the deeper layers of the intestinal wall and later migrate through the intestinal wall. A majority of the patients with advanced colorectal cancer develop liver metastasis during the course of the disease.

Several therapeutic drugs are today used for treatment of CRC. These include Eloxatine® (oxaliplatin), Camptosar® (irinotecan), OncoVAX, Tomudex® (raltitrexed), TS-1, Futulon (doxifluridine) and Xeloda (capecitabine). Several therapeutic products are in late development including Thalomide® (thalidomide), Avastin® (bevacizumab), NeuTrexin® (trimetrexate), Panorex® (edrecolomab) and Erbitux (cetuximab).

The prognosis for the patient is very dependent on the progress of the disease. With no metastasis and localization of the tumor(s) to bowel mucosa the 5-year survival prognosis is 80%, while patients with advanced CRC with distant metastasis have a low (<5%) 5-year survival prognosis.

Accordingly, it is critical to diagnose CRC at an early stage before the disease invades deeper layers of the intestinal wall and before the patients develop liver metastasis. The clinical symptoms of CRC are often non-specific. However, typical symptoms can be discoloured stool (blood in the stool), abdominal pain, weight loss, fever and diarrhoea. The methods used to diagnose CRC include coloscopy, fecal occult blood testing, sigmoidoscopy and double-contrast barium colonography. CT colonography is comparable to colonoscopy for detection of colorectal polyps equal to or larger than 10 mm. The American Cancer Society and others have suggested performing CRC screening of the population or parts of the population. Several clinical studies conclude that screening for CRC is cost effective compared to no screening. Although screening methods for early detection of CRC is available, many patients have CRC diagnosed at a late stage and have poor prognosis. There are several advantages related to the methods used to screen and diagnose CRC today. However, colonoscopy has always a risk of perforation, faecal occult blood testing results in very many false positive results based on other sources of blood, as for example haemorrhoids. No methods, including x-ray methods, are CRC specific and therefore result in many false positive results (e.g. polyps). Existing diagnostic methods for diagnosis of CRC not only result in many false positive results, but the use of these methods also results in many false negative results. None of these methods are useful for safe early diagnosis of CRC at the stage where the disease is superficial. The most specific method might be positron emission tomography (PET) with fluorodeoxyglucose (FDG), but this method is expensive and should be reserved for equivocal cases.

A study of recently published literature on CRC shows that there is a medical need for a cheap, simple and safe method for diagnosis of CRC at an early stage.

U.S. Pat. No. 6,455,688 claims a method for diagnosing CRC by determining the expression of a gene encoding a specific sequence (CJA8).

U.S. Pat. No. 6,326,148 provides a method of screening for colon carcinoma cells in a sample by determining the presence of increased copy number of chromosome 20q.

U.S. Pat. No. 6,316,272 suggests a method of diagnosis of CRC related to a specific nucleic acid sequence.

U.S. Pat. No. 6,187,591 claims a screening test for colorectal cancer whereby a marker is detected in rectal mucus. The marker is detected in the mucus deposited on a support using Schiff's reagent.

U.S. Pat. No. 6,150,100 claims a method for diagnosis of tumors of the gastrointestinal tract such as colorectal tumors based on determination of the genomic instability at 5 selected microsatelite loci.

U.S. Pat. No. 6,149,581 claims a device and method for access to the colon and small bowel of a patient.

U.S. Pat. No. 5,416,025 claims a method for detecting CRC by adding an enzyme to a mucus sample to detect a specific disaccharide marker.

U.S. Pat. No. 5,380,647 claims a test for detecting carcinoembryonic antigen (CEA) in stool. CEA is indicator of the presence of CRC.

U.S. Pat. No. 4,996,298 claims a new method for diagnosis of CRC based on glycoprotein as a marker for CRC.

U.S. Pat. No. 4,857,457 claims a method for detecting the presence of precancer or cancer of the large intestine by assaying the presence of a disaccharide in a mucus sample.

JP II-225800 claims a method for detecting colon cancer using fluorescent material. The method relates to telomerase, however, the method is an in vitro method and does not suggest contrast agents.

As pointed out CRC is still a challenge to diagnose and treat. There is a need for improved diagnostic methods, especially for diagnosis of CRC in an early stage with good reliability. We have surprisingly discovered that the use of optical imaging methods and new contrast agents fulfill these requirements.

### SUMMARY OF THE INVENTION

In view of the needs of the art the present invention provides an optical imaging contrast agent with affinity for an abnormally expressed biological target associated with CRC.

The invention is also described in the claims.

The following definitions will be used throughout the document:

CRC tissue: Any tissue in the colon or rectum that shows changes associated with neoplasia or preneoplasia, and including metastases from colorectal cancer at other sites in the body.

Abnormally expressed target: A target that is either overexpressed, downregulated or mutated in CRC tissue.

Overexpressed target: A receptor, an enzyme or another molecule or chemical entity that is present in a higher amount in CRC tissue than in normal tissue.

Downregulated target: A receptor, an enzyme or another molecule or chemical entity that is present in a lower amount in CRC tissue than in normal tissue.

Mutated target: A protein in CRC tissue that is altered as a result of a germline or sotatic mutation, and including alterations resulting from differential splicing of RNA and changes in post-translational modifications, particularly glycosylation patterns, but not limited to-these types of alterations.

### DETAILED DESCRIPTION OF THE INVENTION

A first aspect of the present invention is an optical imaging contrast agents for imaging of CRC. By the term optical imaging contrast agent, or just contrast agent, we mean a molecular moiety used for enhancement of image contrast in vivo comprising at least one moiety that interacts with light in the ultraviolet, visible or near infrared part of the electromagnetic spectrum.

The contrast agent has affinity for an abnormally expressed target associated with CRC. By abnormally expressed, is meant that the target is either downregulated, mutated or overexpressed. That is, the contrast agent has affinity for a target that is either downregulated, mutated or overexpressed in CRC tissue.

CRC tissue containing a downregulated target is identified by a low amount of bound contrast agent compared to normal tissue. In this situation, the amount of contrast agent should be less than 50% of that in normal tissue, preferably less than 10%.

Targets that are mutated in CRC tissue are identified by lack of binding of a contrast agent that does bind to normal tissue; alternatively, the contrast agent might be directed specifically towards the mutated target, and binding to normal tissue would be minimal. Mutations in CRC-associated genes are often non-random. For instance, more than 90% of mutations in the K-ras gene observed in CRC occur at codon 12 or 13. Somatic mutations in the important adenomatous polyposis coli (APC) gene commonly occur at codons 1309-1311 or codon 1450.

Preferred contrast agents according to the invention have affinity for an overexpressed target associated with CRC. Preferred targets are those targets that are more than 50% more abundant In CRC tissue than in surrounding tissue. More preferred targets are those targets that are more than two times more abundant in CRC tissue than in surrounding tissue. The most preferred targets are those targets that are more than 5 times more abundant in CRC tissue than in surrounding tissue.

Relevant groups of targets are receptors, enzymes, nucleic acids, proteins, lipids, other macromolecules as for example lipoproteins and glycoproteins. The targets may be located in the vascular system, in the extracellular space, associated with cell membranes or located intracellularly.

The following biological targets are preferred targets for contrast agents for optical imaging of CRC:

Adhesion Molecules and Adhesion-Associated Molecules:

Beta-catenin, E-cadherin (CDH1 gene), adenomatous polyposis coli protein (APC), p120-catenin, CD44-standard, CD44-6v, CD44-9v, 67-kDa laminin receptor, P-cadherin and integrins, such as αᵥβ₃. and αᵥβ₆.

Antigens:

Human leukocyte antigen-B18 and human leukocyte antigen-DQ5, tissue polypeptide antigen (TPA) or tissue polypeptide-specific antigen (TPS), Small intestinal mucin antigen (SIMA), CA15.3, CA 19-9, CA 72-4, CYFRA 21-1, CAM 17.1, CEA, TPS, CA 72-4, MUC-1, tumour-associated antigen L6, HLA-A, CA-195, CA-242, beta HCG, AFP, CA125.

Enzymes:

alpha-methylacyl-CoA racemase, aminopeptidase N/CD13, carcinogen metabolising enzymes, arachidonic acid metabolism, enzymes responsible for polyamine metabolism, CDC25B phosphatase, COX-1, cyclooxygenase-2 (COX-2), cytochrome P450 2A6 (CYP2A6), glutathione S-transferase, gamma-glutamylcysteine synthetase (gamma-GCS) and DT-diaphorase, guanylyl cyclase C, matrix metalloproteinases and their inhibitors (especially MMP-2, MMP-7, MMP-9, stromelysin-3 and MT1-MMP (=MMP 14)), mitochondrial aspartate-aminotransferase, phosphoglucomutase, plasminogen-related molecules, thymidylate synthase, tumour-associated trypsin inhibitor (TATI), u-PA, prostaglandin E synthase and cathepsins, typically cathepsin B and human aspartyl (asparginyl) beta-hydroxylase (HAAH),

Signal Molecules and Receptors:

Beta-HCG, c-erbB, and VEGF, c-Myc, gastrin, CCK(B)-R, gastrin, bradeion (septin family gene), WNT7A, WNT7B, insulin-like growth factor 2, benzodiazepine receptor, Her-2, VEGF receptors, EGF receptors, IL-8, CXCR1, CXCR2, Urokinase Plasminogen Activator Receptor (u-PAR), urokinase receptor associated protein (u-PARAP/Endo 180), c-met and angiotensin1 -receptor (AT1R).

Tumour Suppressor Proteins, Oncogenes, Apoptosis-Related Proteins:

Adenomatous polyposis coli protein (APC), bax, Bcl-2, beta-catenin/T cell factor-4 (Tcf-4), groucho proteins, proteins in K-ras cascade, nm23, p53, K-ras, Deleted in Colorectal Cancer (DCC), c-erbB2, survivin, SMAD2, SMAD4.

Others:

L-plastin, the human homologue of yeast ribosomal protein S28, the B-cell translocation gene, AXIN2, chromogranin A, synaptophysin, syntaxin1, VAMP2, SNAP25, alpha/beta-SNAP, clusterin (apolipoprotein j), ITF-2, PPARdelta, cystatin-like metastasis-associated protein, EBP50, etheno (epsilon)-DNA adducts (e.g., via trans-4-hydroxy-2-nonenal), keratin 5, Ki-67, Mib-1, proliferating cell nuclear antigen, osteopontin, p27 (kip-1), proliferating cell nuclear antigen (PCNA), WAF1, p34cdc2, cyclins B1 and D1, SBA2, sigma B3 protein, transcription factor nuclear factor-kappa beta (NF-kappaB) and hypoxia-inducible factor.

Among the more preferred targets for contrast agents for optical imaging of CRC are: COX-2, beta-catenin, E-cadherin, P-cadherin, various kinases, Her-2, MMPs, cyclins, P53, thymidylate synthase, VEGF receptors, EGF receptors, K-ras, adenomatous polyposis coli protein, cathepsin B, uPAR, c-met, mucins and gastrin receptors.

The most preferred targets for contrast agents for optical imaging of CRC are: c-met, MMP-14, COX-2, beta-catenin and cathepsin B.

Generally, any targets that have been identified as possible targets for agents for treatment of CRC are potential targets also in optical imaging.

The preferred contrast agents are molecules with relatively low molecular weights. The molecular weight of preferred contrast agents is below 10000 Daltons, more preferably below 7000 Daltons.

The contrast agents are preferably comprised of a vector that has affinity to an abnormally expressed target in CRC tissue, and an optical reporter. Thus viewed from one aspect the present invention provides a contrast agent of formula I:
V-L-R (I)

wherein V is one or more vector moieties having affinity for one or more abnormally expressed target in CRC tissue, L is a linker moiety or a bond and R is one or more reporter moieties detectable in optical imaging.

The vector has the ability to direct the contrast agent to a region of CRC. The vector has affinity for the abnormally expressed target and preferably binds to the target. The reporter is detectable in an optical imaging procedure and the linker must couple vector to reporter, at least until the reporter has been delivered to the region of CRC and preferably until the imaging procedure has been completed.

The vector can generally be any type of molecules that have affinity for the abnormally expressed target. The molecules should be physiologically acceptable and should preferably have an acceptable degree of stability. The vectors can for instance be selected from the following group of compounds: peptides, peptoids/peptidomimetics, oligonucleotides, oligosaccharides, lipid-related compounds like fatty acids, traditional organic drug-like small molecules, synthetic or semi-synthetic, and derivatives and mimetics thereof. When the target is an enzyme the vector may comprise an inhibitor of the enzyme or an enzyme substrate. The vector of the contrast agent preferably has a molecular weight of less than 4500 Daltons and more preferably less than 2500 Daltons.

Contrast agents having affinity for more than one abnormally expressed target related to the disease is an aspect of the invention. Such contrast agents can comprise two or more different vectors or molecular subunits that target two or more different abnormally expressed targets.

Another possibility according to the present invention is that the contrast agent comprises one vector that is able to bind to more than one abnormally expressed target in CRC tissue.

A contrast agent according to the present invention can also comprise more than one vector of same chemical composition that bind to the abnormally expressed biological target.

Some receptors are unique to endothelial cells and surrounding tissues. Examples of such receptors include growth factor receptors such as VEGF and adhesion receptors such as the integrin family of receptors. Peptides comprising the sequence arginine-glycine-aspartic acid (RGD) are known to bind to a range of integrin receptors. Such RGD-type peptides constitute one group of vectors for targets associated with CRC.

Below are some examples of vectors having affinity for CRC related abnormally expressed targets:

Vectors for COX-2:

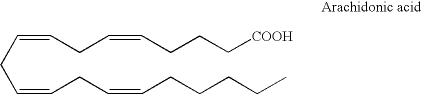

Arachidonic acid is the endogenous substrate for COX-2.

Other vectors for COX-2 are exogenous compounds that bind to COX-2, for example so-called COX-2 inhibitors. The chemical classes of the main COX-2 inhibitors are shown in WO 02/07721.

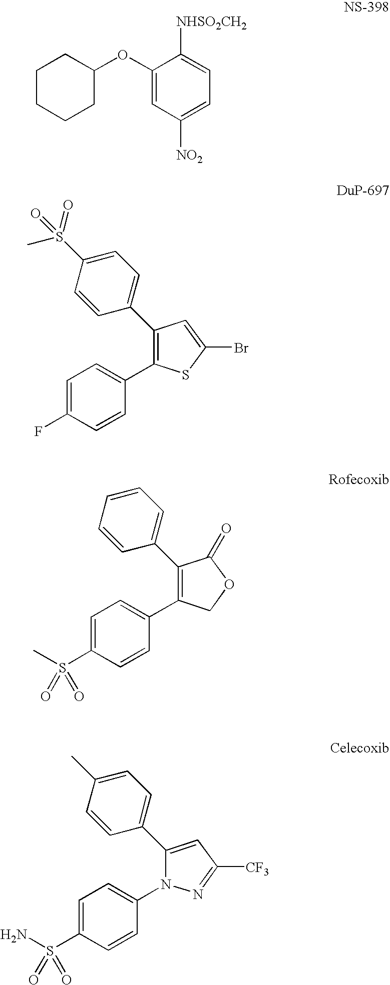

Vectors for MMP-7:

Peptide sequence: Cys-Gly-Pro-Leu-Gly-Leu-Leu-Ala-Arg-OH

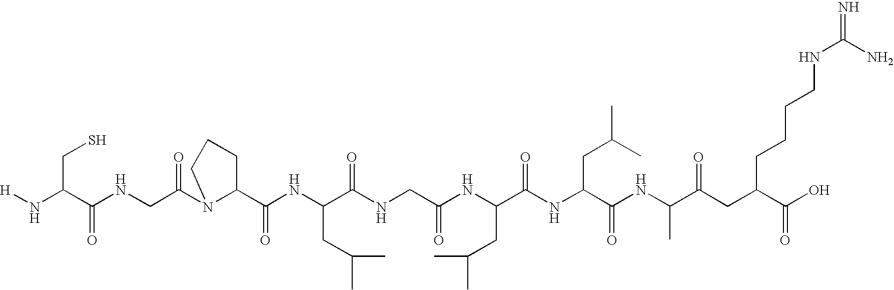

Vector for Benzodiazepine Receptor:

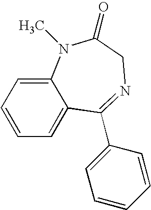

Vectors for Integrins: RGD-Type Peptides Having Affinity for αᵥβ₃. and αᵥβ₅:

c[-Asp-D-Phe-Lys-Arg-Gly-]

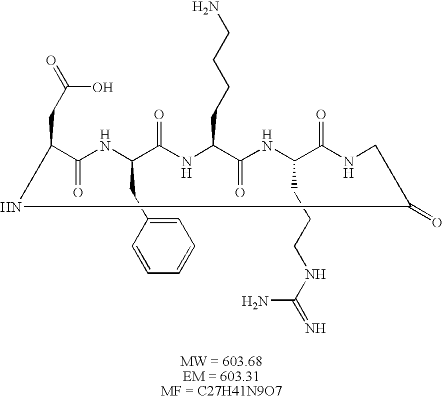

A wide variety of linkers can be used. The linker component of the contrast agent is at its simplest a bond between the vector and the reporter moieties. In this aspect the reporter part of the molecule is directly bound to the molecule sub-unit that binds to the abnormally expressed target. More generally, however, the linker will provide a mono- or multi-molecular skeleton covalently or non-covalently linking one or more vectors to one or more reporters, e.g. a linear, cyclic, branched or reticulate molecular skeleton, or a molecular aggregate, with in-built or pendant groups which bind covalently or non-covalently, e.g. coordinatively, with the vector and reporter moieties. The linker group can be relatively large in order to build into the contrast agent optimal size or optimal shape or simply to improve the binding characteristics for the contrast agent to the abnormally expressed target in CRC tissue.

Thus, linking of a reporter unit to a desired vector may be achieved by covalent or non-covalent means, usually involving interaction with one or more functional groups located on the reporter and/or vector. Examples of chemically reactive functional groups which may be employed for this purpose include amino, hydroxyl, sulfhydroxyl, carboxyl and carbonyl groups, as well as carbohydrate groups, vicinal diols, thioethers, 2-aminoalcohols, 2-aminothiols, guanidinyl, imidazolyl and phenolic groups.

The reporter is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye part of the contrast agent can be any dye that interacts with light in the electromagnetic spectrum with wavelengths from the ultraviolet light to the near infrared. Preferably, the contrast agent of the invention has fluorescent properties.

Preferred organic chromophoric and fluorophoric reporters include groups having an extensive delocalized electron system, eg. cyanines, merocyanines, indocyanines, phthalocyanines, naphthalocyanines, triphenylmethines, porphyrins, pyrilium. dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include fluorescein, sulforhodamine 101 (Texas Red), rhodamine B, rhodamine 6G, rhodamine 19, indocyanine green, Cy2, Cy3B, Cy3.5, Cy5, Cy5.5, Cy7, Cy7.5, Marina Blue, Pacific Blue, Oregon Green 488, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. The cyanine dyes are particularly preferred.

Particularly preferred are dyes which have absorption maxima in the visible or near infrared region, between 400 nm and 3 μm, particularly between 600 and 1300 nm. The contrast agents according to the invention can comprise more than one dye molecular sub-unit. These dye sub-units can be similar or different from a chemical point of view. Preferred contrast agents have less than 6 dye molecular sub-units.

Several relevant targets for CRC are enzymes. A contrast agent for optical imaging of CRC for targeting an enzyme can be an enzyme contrast agent substrate that can be transformed to a contrast agent product possessing different pharmacokinetic and/or pharmacodynamic properties from the contrast agent substrate. This embodiment of the invention provides contrast agent substrates having affinity for an abnormally expressed enzyme,.wherein the contrast agent substrate changes pharmacodynamic and/or pharmacokinetic properties upon a chemical modification into a contrast agent product in a specific enzymatic transformation, and thereby enabling detection of areas of disease upon a deviation in the enzyme activity from the normal. Typical differences in pharmacodynamic and/or pharmacokinetic properties can be binding properties to specific tissue, membrane penetration properties, protein binding and solubility properties.

Alternatively, if the abnormally expressed target for diagnosis of CRC is an enzyme, the contrast agent for optical imaging can be a dye molecule that directly binds to the enzyme. The contrast agent will have affinity for the abnormally expressed enzyme, and this may be used to identify tissue or cells with increased enzymatic activity.

In a further aspect of the invention, the contrast agent changes dye characteristics as a result of an enzymatic transformation. For example, a fluorescent dye reporter of the contrast agent is quenched (no fluorescence) by associated quencher groups, until an enzymatic cleavage takes place, separating the dye from the quencher groups and resulting in fluorescence at the site of the abnormally expressed enzyme.

Another aspect of this part of the invention is that the dye may change colour, as e.g. a change in absorption and/or emission spectrum, as a result of an enzymatic transformation.

If the abnormally expressed target for diagnosis of CRC is a receptor or another non-catalytical target, the contrast agent for optical imaging can bind directly to the target and normally not change the dye characteristics.

Another aspect of the invention is contrast agents for optical imaging of CRC characterized by having affinity for more than one abnormally expressed target related to the disease. Such contrast agents can have two or more different vectors or molecular subunits that target two or more different abnormally expressed targets.

The preferred contrast agents of the present invention are soluble in water. This means that the preferred contrast agents have a solubility in water at pH 7.4 of at least 1 mg/ml.

The contrast agents of the present invention can be identified by random screening, for example by testing of affinity for abnormally expressed targets of a library of dye labelled compounds either prepared and tested as single compounds or by preparation and testing of mixture of compounds (a combinatorial approach).

The contrast agents of the present invention can also be identified by use of technology within the field of intelligent drug design. One way to perform this is to use computer-based techniques (molecular modelling or other forms of computer-aided drug design) or use of knowledge about natural and exogenous ligands (vectors) for the abnormally expressed targets. The sources for exogenous ligands can for example be the chemical structures of therapeutic molecules for targeting the same target. One typical approach here will be to bind the dye chemical sub-unit to the targeting vector so that the binding properties of the vector are not reduced. This can be performed by linking the dye at the far end away from the pharmacophore centre (the active targeting part of the molecule). Alternatively, random screening may be used to identify suitable vectors before labelling with a reporter.

The contrast agents of the invention are preferably not endogenous substances alone. Some endogenous substances, for instance estrogen, have certain fluorescent properties in themselves, but they are not likely to be sufficient for use in optical imaging. Endogenous substances combined with an optical reporter however, falls within the contrast agents of the invention.

The contrast agents of the invention are intended for use in optical imaging. Any method that forms an image for diagnosis of disease, follow up of disease development or for follow up of disease treatment based on interaction with light in the electromagnetic spectrum from ultraviolet to near-infrared radiation falls within the term optical imaging. Optical imaging further includes all methods from direct visualization without use of any device and use of devices such as various scopes, catheters and optical imaging equipment, for example computer based hardware for tomographic presentations. The contrast agents will be useful with optical imaging modalities and measurement techniques including, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

Examples of contrast agents for optical imaging of CRC according to the invention are shown below:

Contrast Agents for Mapping of COX-2:

1.

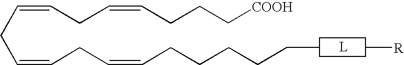

Wherein arachidonic acid, the endogenous substrate for COX-2, is coupled to a reporter (R) via a linker (L).

2.

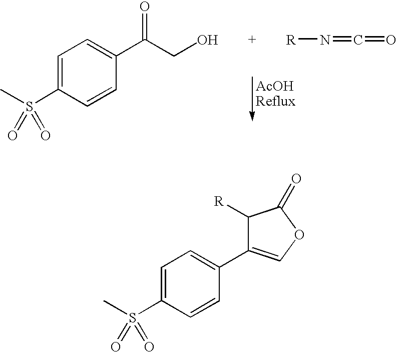

Wherein a COX-2 inhibitor derivative is linked to a reporter. R is any reporter according to the present invention; for example fluorescein, and L is a linker.

Contrast Agent for Mapping of Matrix Metalloproteinase:

The vector peptide is linked to e.g. fluorescein (R) through a linker (L):

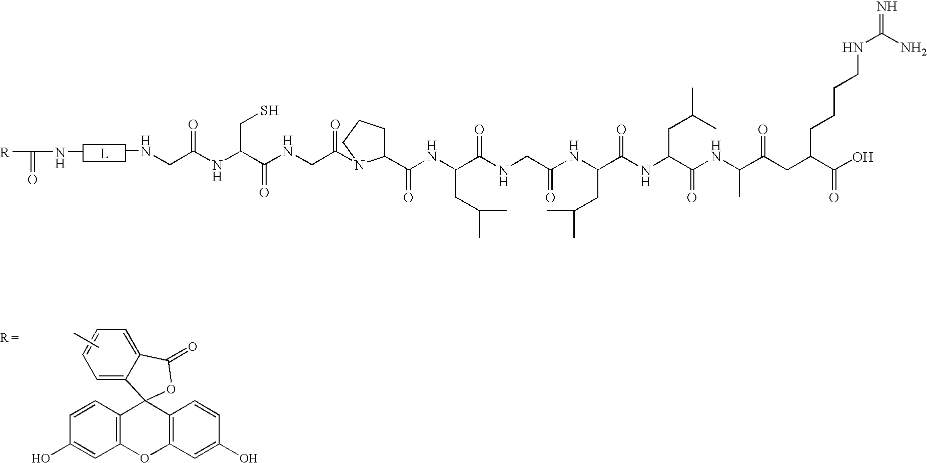

Contrast Agent with Affinity for Benzodiazepine Receptor:

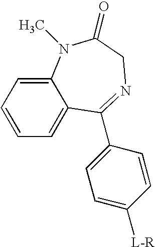

Wherein L is a linker and R is one of the mentioned reporters.

A further embodiment is use of contrast agents of the invention for optical imaging of CRC, that is, for diagnosis of CRC, for use in follow up the progress in CRC development or for follow up the treatment of CRC. In the context of this invention, diagnosis includes screening of selected populations, early detection, biopsy guidance, characterisation, staging, grading, therapy efficacy monitoring, long-term follow-up of relapse and surgical guidance.

Still another embodiment of the invention is a method of optical imaging for diagnosis of CRC using the contrast agents as described.

Still another embodiment of the invention is a method of optical imaging for diagnosis, to follow up the progress of CRC development and to follow up the treatment of CRC, using the contrast agents as described.

One aspect of these methods is to administer the present contrast agents and follow the accumulation and elimination directly visually during surgery. Another aspect of these methods is to administer the present contrast agents and perform visual diagnosis through a colonoscope.

Still another aspect of the present invention is to administer the present contrast agents and perform the image diagnosis using computerized equipment as for example a tomograph.

Still another embodiment of the invention is use of a contrast agent as described for the manufacture of a diagnostic agent for use in a method of optical imaging of CRC involving administration of said diagnostic agent to an animate body and generation of an image of at least part of said body.

Still another embodiment of the invention is pharmaceutical compositions comprising one or more contrast agents as described or pharmaceutically acceptable salts thereof for optical imaging for diagnosis of CRC, for follow up progress of CRC development or for follow up the treatment of CRC. The diagnostic agents of the present invention may be formulated in conventional pharmaceutical or veterinary parenteral administration forms, e.g. suspensions, dispersions, etc., for example in an aqueous vehicle such as water for injections. Such compositions may further contain pharmaceutically acceptable diluents and excipients and formulation aids, for example stabilizers, antioxidants, osmolality adjusting agents, buffers, pH adjusting agents, etc. The most preferred formulation is a sterile solution for intravascular administration or for direct injection into area of interest. Where the agent is formulated in a ready-to-use form for parenteral administration, the carrier medium is preferably isotonic or somewhat hypertonic.

The dosage of the optical diagnostic agents of the invention will depend upon the clinical indication, choice of contrast agent and method of administration. In general, however dosages will be between 10 μg and 5 grams for an adult human;

While the present invention is particularly suitable for methods involving parenteral administration of the contrast agent, e.g. into the vasculature or directly into an organ of muscle tissue, intravenous administration being especially preferred, it is also applicable where administration is not via a parenteral route, e.g. where administration is transdermal, nasal, sub-lingual or is into an externally voiding body cavity, e.g. the colon, rectum or bladder. The present invention is deemed to extend to cover such administration.

The following examples are illustrative only and not intended to be limiting. Other features and advantages of the invention will be apparent from the detailed description and from the claims.

### EXAMPLES

### Example 1

### Contrast Agent for Mapping of COX-2 Activity. Synthesis of COX-2 Ligand Coupled to Fluorescein

Step 1

2-Hydroxy-1-(4-methanesulfonylphenyl)ethanone is prepared from 2-bromo-1-(4-methanosulfonylphenyl)ethanone according to C. Puig et al in J. Med. Chem 2000,43, 214-223.

Step 2

A solution of 2-hydroxy-1-(4-methanosulfonylphenyl)ethanone (1.50 g, 7 mmol) and fluorescein isocyanate isomer 1 (2.72 g, 7 mmol) is heated in DMF at 120° C. for 5 hours.

The mixture is cooled, DMF evaporated off and acetic acid (25 ml) is added. The mixture is refluxed for 10 hours. The acetic acid is evaporated and the resulting mixture is purified on silica using chloroform/methanol as eluent.

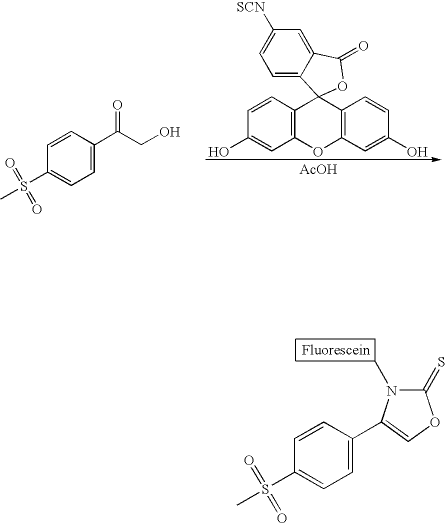

### Example 2

### Contrast Agent for Mapping of Matrix Metalloproteinase (MMP). Synthesis of fluorescein-Cys-Gly-Pro-Leu-Gly-Leu-Leu-Ala-Arg-OH Linker Conjugate

Step 1

The peptide component was synthesised on an ABI 433A automatic peptide synthesiser starting with Fmoc—Arg(Pmc)—wang resin on a 0.1 mmol scale using 1 mmol amino acid cartridges. The amino acids were pre-activated using HBTU before coupling. An aliquot of the peptide resin was then transferred to a clean round bottom flask an N-methyl morpholine (1 mmol) in DMF (5 ml) added followed by chloroacetyl chloride (1 mmol). The mixture was gently shaken until Kaiser test negative. The resin was extensively washed with DMF.

Step 2

5(6)—carboxyfluorescein (188 mg, 0.5 mmol) and dicyclohexylcarbodiimide (113 mg, 0.55 mmol) are dissolved in DMF (20 ml). The mixture is stirred for 2 hours and cooled to 0° C. A solution of hexamethylenediamide (116 mg, 1 mmol) and DMAP (30 mg) in DMF is added and the mixture is stirred at ambient temperature for 72 hours. The solution is evaporated and the conjugate between carboxyfluorescein and hexamethylene-amine is isolated as monoamide by chromatography (silica, chloroform and methanol).

Step 3

The resin from step 1 is suspended in DMF (5 ml) and amide-amine conjugate from step 2 (0.5 mmol) pre-dissolved in DMF (5 ml) containing triethylamine (0.5 mmol) is added. The mixture is heated to 50° C. for 16 hours then excess reagents filtered off, following extensive washing with DMF, DCM and diethyl ether then air drying. The product is treated with TFA containing TIS (5%), H_{z}0 (5%), and phenol (2.5%) for 2 hours.

Excess TFA is removed in vacuo and the peptide is precipitated by the addition of diethyl ether. The crude peptide conjugate is purified by preparative HPLC C C-18, acetonitril, TFA, water).

### Example 3

### Contrast Agent for Binding to Benzodiazepine Receptor

Step 1

Nitrazepam is reduced to the corresponding 7-aminonitrazepam using standard condition zink in aqueous hydrochloric acid, catalytic hydrogenation or other reduction agents.

Step 2

5(6) Carboxyfluorescein (1 mmol) and dicyclohexylcarbodiimide (1 mmol) are dissolved in DMF (30 ml). The mixture is stirred for 2 hours at ambient temperature. A solution of 7-aminonitrazepam (1 mmol) and DMAP (20 mg) in DMF (10 ml) is added and the mixture is evaporated and the conjugate between 7-aminonitrazepam and 5(6) carboxyfluorescein is isolated by chromatography (silica, chloroform/methanol).

### Example 4

### Contrast Agent for Binding to p53 Oncoprotein

Step 1 Synthesis of 2,2-bis(hydroxymethyl)-1-aza-bicyclo[2,2,2,]octan-3-one. 3-quinuclidinone hydrochloride (Aldrich Q 190-5) (1 mmol) is dissolved in methanol-water (1:1, 30 ml). An aqueous solution of formaldehyde (37%, 2.5 mmol) and sodium hydroxide (1.5 mmol) are added. The mixture is stirred for 12 hours at 50° C. The solvents are evaporated and the title compound isolated as free base using flash chromatography (silica, ethylacetate/chloroform, hexane).

Step 2.

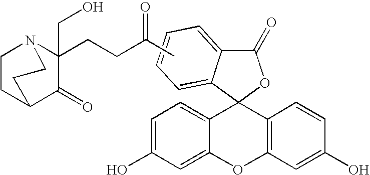

5(6)-carboxyfluorescein (0.1 mmol) and dicyclohexyl carbodiimide (0.11 mmol) are dissolved in DMF. The mixture is stirred for 3 hours and cooled to 0° C. A solution of 2,2-bis(hydrozymethyl)-1-azabicyclo[2,2,2]octane-3-one (0.5 mmol) and DMAP (10 mg) in DMF is added and the mixture is stirred at ambient temperature for 72 hours. The solution is evaporated and the contrast agent is isolate by flash chromatography (silica, ethyl acetate/hexane).

### Example 5

### Contrast Agent with Affinity for Integrins: RGD Peptide Linked to Cy5.5

Step 1. Assembly of Amino Acids

The peptide sequence Asp-D-Phe-Lys-Arg-Gly was assembled on an Applied Biosystems 433A peptide synthesizer starting with 0.25 mmol Fmoc-Gly-SASRIN resin. An excess of 1 mmol pre-activated amino acids (using HBTU; O-Benzotriazol-1-yl-N,N,N′,N′-tetramethyluronium hexafluorophosohate) was applied in the coupling steps. The cleavage of the fully protected peptide from the resins was carried out by treatment of the resin with three portions of 35 mL of 1% trifluoroacetic acid (TFA) in dichloromethane (DCM) for 5 minutes each. The filtrates containing the peptide was immediately neutralised with 2% piperidine in DCM. The organics were extracted with water (3×100 mL), dried with MgSO₄ and evaporated in vacuo. Diethyl ether was added to the residue and the precipitate washed with ether and air-dried affording 30 mg of crude protected peptide. The product was analysed by analytical HPLC (conditions: Gradient, 20-70% B over 10 min where A=H₂O/0.1% TFA and B=CH₃CN/0.1% TFA; flow, 2 mL/min; column, Phenomenex Luna 3μ 5×4.6 mm; detection, UV 214 nm; product retention time 7.58 min). Further product characterisation was carried out using electrospray mass spectrometry (MH⁺ calculated, 1044.5; MH⁺ found, 1044.4).

Step 2. N-C Cyclisation

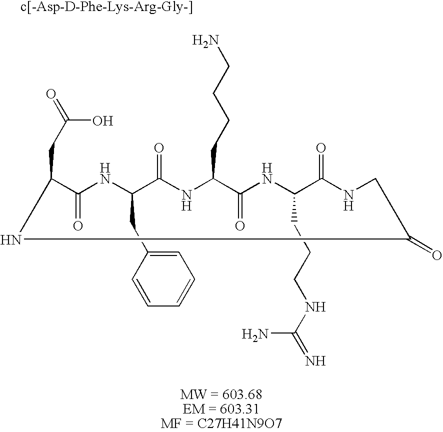

30 mg of the fully protected peptide, 16 mg of PyAOP, 4 mg of HOAt and 6 μL of N-methylmorpholine (NMM) were dissolved in dimethylformamide/DCM (1:1) and stirred over night. The mixture was evaporated in vacuo and diethyl ether added to the residue. The precipitate was washed with ether and air-dried. The crude cyclic fully protected peptide was treated with a solution of 25 mL TFA containing 5% water, 5% triisopropylsilane and 2.5% phenol for two hours. TFA was evaporated in vacuo and diethyl ether added to the residue. The precipitate was washed with ether and air-dried. Purification by preparative RP-HPLC (0-30% B over 40 min, where A=H₂O/0.1% TFA and B=CH₃CN/0.1% TFA, at a flow rate of 10 mL/min on a Phenomenex Luna 5μ C18 250×21.20 mm column) of the crude material afforded 2.3 mg pure product peptide. The pure product was analysed by analytical HPLC (conditions: Gradient, 0-15% B over 10 min where A=H₂O/0.1% TFA and B=CH₃CN/0.1% TFA; flow, 2 mL/min; column, Phenomenex Luna 3μ 5×4.6 mm; detection, UV 214 nm; product retention time 6.97 min). Further product characterisation was carried out using electrospray mass spectrometry (MH⁺ calculated, 604.3; MH⁺ found, 604.4).

Step 3. Conjugation of Cy5.5 to RGD Peptide

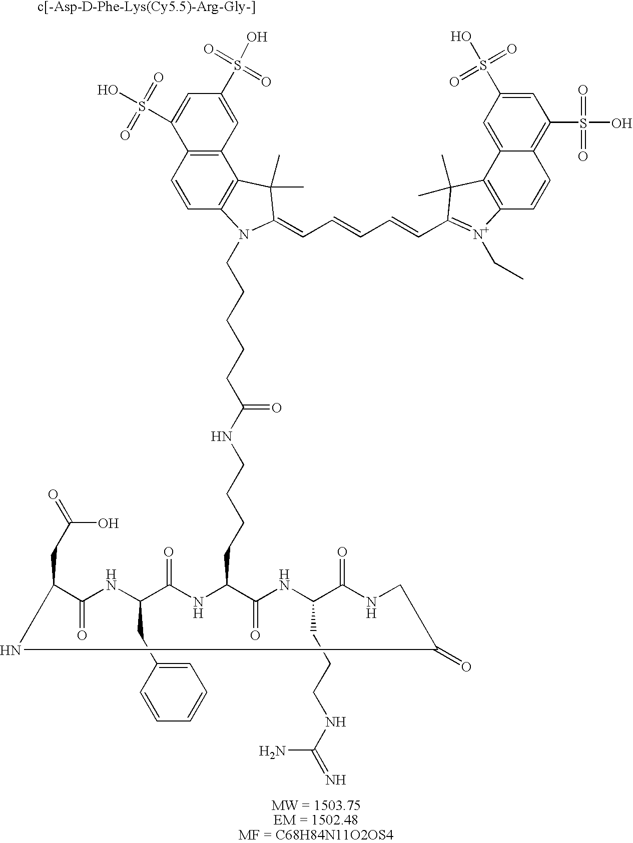

0.6 mg of the RGD peptide, 1:7 mg of Cy5.5 mono NHS ester and 5 μL of NMM were dissolved in 1 mL of dimethylformamide (DMF) and the reaction mixture stirred for 2 hrs. Diethyl ether was added to the DMF solution and the blue precipitate washed with diethyl ether and air-dried affording 0.7 mg of crude RGD peptide conjugated to Cy5.5.The pure product was analysed by analytical HPLC (conditions: Gradient, 5-50 % B over 10 min where A=H₂O/0.1% TFA and B=CH₃CN/0.1% TFA; flow, 0.3 mL/min; column, Phenomenex Luna 3μ5×2 mm; detection, UV 214 nm; product retention time 8.32 min). Further product characterisation was carried out using electrospray mass spectrometry (MH⁺ calculated, 1502.5; MH⁺ found, 1502.6).

### Example 6

### Synthesis of Losartan Derivatised with Cy5.5—Contrast Agent for Binding to AT1R

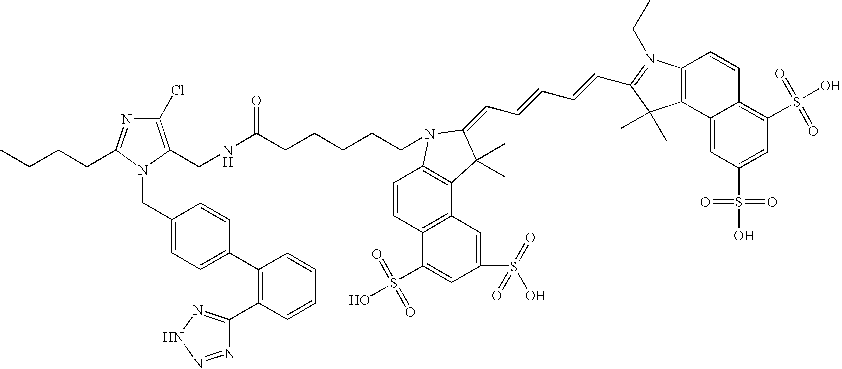

a) Replacement of Losartan Hydroxyl Group by Azide

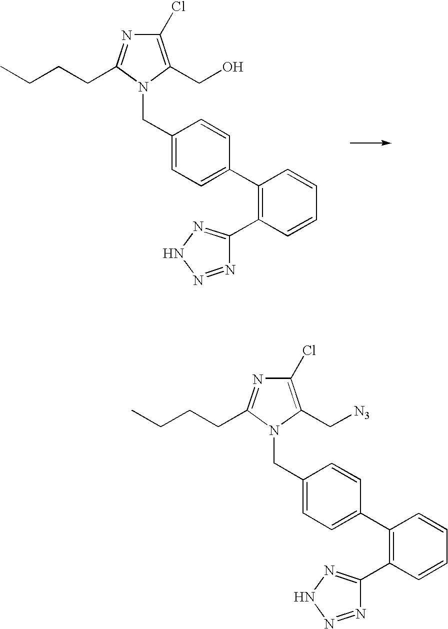

To a stirred suspension of Losartan (MSD, 0.423 g, 1.00 mmol) and diphenylphosphoryl azide (Aldrich, 0.259 ml, 1.20 mmol) in tetrahydrofuran (8 ml) was added DBU (0.329 ml, 2.20 mmol). After stirring overnight water/acetonitrile (1:1, 4.8 ml) was added and the mixture was filtered. After addition of neat TFA (to pH 2) the mixture was purified by preparative HPLC (column Phenomenex Luna C18(2) 5 μm 21.2×250 mm, solvents: A=water/0.1% TFA and B=acetonitrile/0.1% TFA; gradient 35-45% B over 60 min; flow 10.0 ml/min, UV detection at 214 nm) in several runs to give.99 mg (22%) of the product as white crystals after lyophilisation. Analysis by LC-MS (column Phenomenex Luna C18(2) 3 μm 50×4.60 mm, solvents: A=water/0.1% TFA and B=acetonitrile/0.1% TFA; gradient 20-80% B over 10 min; flow 1 ml/min, UV detection at 214 nm, ESI-MS) gave a peak at 7.3 minutes with m/z 448.1 (MH⁺) corresponding to the structure.

b) Reduction of the Azide Group to Amino Function

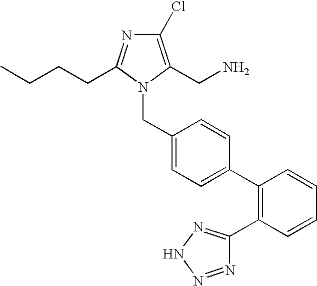

To a solution of compound from a) (5.0 mg, 0.011 mmol) in methanol (3 ml) was added Pd/C (Koch-Light, ca 10 mg). The mixture was stirred under hydrogen (1 atm) for 10 min, filtered and concentrated. The residue was used in the next step without further work up. LC-MS analysis (column Phenomenex Luna C18(2) 3 μm 50×4.60 mm, solvents: A=water/0.1% TFA and B=acetonitrile/0.1% TFA; gradient 20-80% B over 10 min; flow 1 ml/min, UV detection at 214 nm, ESI-MS) gave a peak at 1.9 minutes with m/z 422.2 (MH⁺) corresponding to the amine.

c) Conjugation of Cy5.5

To a solution of losartanamine from b) (0.5 mg, 1 μmol) in DMF (0.2 ml) were added Cy5.5-NHS (Amersham Biosciences; 1 mg, 1 μmol) and N-methylmorpholine (1 μl, 9 μmol). The reaction mixture was stirred overnight Formation of losartan-Cy5.5 conjugate was confirmed by MS analysis giving m/z at 1320.6 (M⁺), expected 1320.3.

### Example 7

### Synthesis of 3-[(4′-Fluorobiphenyl-4-sulfonyl)-(1-hydroxycarbamoylcyclopentyl)amino]Propionic acid (compound A) Derivatised with Cy5.5—Contrast Agent for Binding to MMP

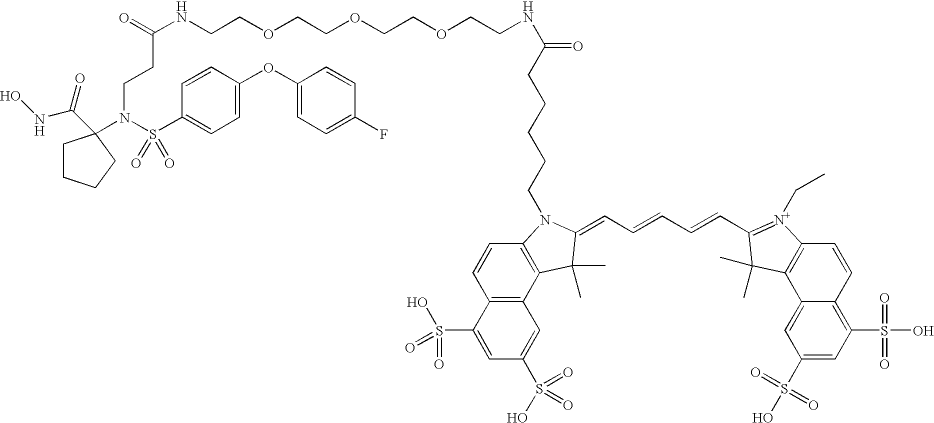

a) 1.11-Diazido-3,6,9-trioxaundecane

A solution of dry tetraethylene glycol (19.4 g, 0.100 mol) and methanesulphonyl chloride (25.2 g, 0.220 mol) in dry THF (100 ml) was kept under argon and cooled to 0° C. in an ice/water bath. To the flask was added a solution of triethylamine (22.6 g, 0.220 mol) in dry THF (25 ml) dropwise over 45 min. After 1 hr the cooling bath was removed and stirring was continued for 4 hrs. Water (60 ml) was added. To the mixture was added sodium hydrogencarbonate (6 g, to pH 8) and sodium azide (14.3 g, 0.220 mmol), in that order. THF was removed by distillation and the aqueous solution was refluxed for 24 h (two layers formed). The mixture was cooled and ether (100 ml) was added. The aqueous phase was saturated with sodium chloride. The phases were separated and the aqueous phase was extracted with ether (4×50 ml). Combined organic phases were washed with brine (2×50 ml) and dried (MgSO₄). Filtration and concentration gave 22.1 g (91%) of yellow oil. The product was used in the next step without further purification.

b) 11-Azido-3,6,9-trioxaundecanamine

To a mechanically, vigorously stirred suspension of 1,11-diazido-3,6,9-trioxaundecane (20.8 g, 0.085 mol) in 5% hydrochloric acid (200 ml) was added a solution of triphenylphosphine (19.9 g, 0.073 mol) in ether (150 ml) over 3 hrs at room temperature. The reaction mixture was stirred for additional 24 hrs. The phases were separated and the aqueous phase was extracted with dichloromethane (3×40 ml). The aqueous phase was cooled in an ice/water bath and pH was adjusted to ca 12 by addition of KOH. The product was extracted into dichloromethane (5×50 ml). Combined organic phases were dried (MgSO₄). Filtration and evaporation gave 14.0 g (88%) of yellow oil. Analysis by MALDI-TOF mass spectroscopy (matrix: □-cyano-4-hydroxycinnamic acid) gave a M+H peak at 219 as expected. Further characterisation using ¹H (500 MHz) and ¹³C (125 MHz) NMR spectroscopy verified the structure.

c) Linking compound A to PEG(4)-N₃

To a solution of compound A (CP-471358, Pfizer, 41 mg, 87 μmol) in DMF (5 ml) were added 11-azido-3,6,9-trioxaundecanamine (19 mg, 87 μmol), HATU (Applied Biosystems, 33 mg, 87 μmol) and DIEA (Fluka, 30 μl, 174 μmol). After one hour reaction time the mixture was concentrated and the residue was purified by preparative HPLC (column Phenomenex Luna C18(2) 5 μm 21.2×250 mm, solvents: A=water/0.1% TFA and B=acetonitrile/0.1% TFA; gradient 30-60% B over 60 min; flow 10.0 ml/min, UV detection at 214 nm), giving 33.9 mg (59%) of product after lyophilisation. LC-MS analysis (column Phenomenex Luna C18(2) 3 μm 50×4.60 mm, solvents: A=water/0.1% TFA and B=acetonitrile/0.1% TFA; gradient 20-100% B over 10 min; flow 1 ml/min, UV detection at 214 nm, ESI-MS) gave a peak at 4.88 min with m/z 667.4 (MH⁺) as expected.

d) Svnthesis of Compound A-PEG(4)-NH₂

To a solution of the PEG(4)-N₃ compound from c) (4.7 mg, 7.0 μmol) in methanol (4 ml) was added Pd/C (Koch-Light, ca 10 mg) added. The mixture was stirred at room temperature under hydrogen atmosphere (1 atm) for 10 min. The mixture was filtered and concentrated. LC-MS analysis (column Phenomenex Luna C18(2) 3 μm 50×4.60 mm, solvents: A=water/0.1% TFA and B=acetonitrile/0.1% TFA; gradient 20-100% B over 10 min; flow 1 ml/min, UV detection at 214 nm, ESI-MS) gave a peak at 4.17 min with m/z 641.4 (MH⁺) as expected. The product was used directly in the next step without further purification.

e) Conjugation of Cy 5.5

To a solution of the amine from d) (1.0 mg, 1.5 μmol) in DMF (0.2 ml) was added Cy 5.5-NHS (Amersham Biosciences, 1.0 mg, 1.0 μmol) and N-methylmorpholine (1 μl, 9 μmol). The reaction mixture was stirred for 48 h. MS analysis of the solution gave a spectrum showing starting material and the conjugated product at m/z 1539.7 (M⁺⁾, expected 1539.4.

What is claimed is:

1-12. (canceled)

13. An optical imaging contrast agent with affinity for an abnormally expressed biological target associated with CRC, wherein the contrast agent has a molecular weight below 10000 Daltons.

14. A contrast agent as claimed in claim 13 of formula I
V-L-R (I)
wherein V is one or more vector moieties having affinity for an abnormally expressed target in CRC, L is a linker moiety or a bond and R is one ore more reporter moieties detectable in optical imaging.

15. A contrast agent as claimed in claim 14 wherein R is a cyanine dye.

16. A contrast agent as claimed in claim 13 wherein the target is a receptor or a non-catalytical target.

17. A contrast agent as claimed in claim 13 comprising a contrast agent substrate, wherein the target is an abnormally expressed enzyme, such that the contrast agent changes pharmacodynamic properties and/or pharmacokinetic properties upon a chemical modification from a contrast agent substrate to a contrast agent product upon a specific enzymatic transformation.

18. A contrast agent as claimed in claim 17 wherein the contrast agent changes binding properties to specific tissue, membrane penetration properties, protein binding or solubility properties upon the chemical modification.

19. A contrast agent as claimed in claim 13 having affinity for any of the targets selected from COX-2, beta-catenin, E-cadherin, P-cadherin, kinases, Her-2, MMPs, cyclins, P53, thymidylate synthase, VEGF receptors, EGF receptors, K-ras, adenomatous polyposis coli protein, cathepsin B, uPAR, c-met, mucins and gastrin receptors.

20. A contrast agent as claimed in claim 14 wherein V is selected from peptides, peptoid moieties, oligonucleotides, oligosaccharides, lipid-related compounds and traditional organic drug-like small molecules.

21. A contrast agent as claimed in claim 20 wherein V is a peptide.

22. A pharmaceutical composition for optical imaging for diagnosis of CRC, for follow up of progress of CRC development or for follow up of treatment of CRC, comprising a contrast agent as defined in claim 13 together with at least one pharmaceutically acceptable carrier or excipient.

23. A contrast agent as claimed in claim 13 for the manufacture of a diagnostic agent for use in a method of optical imaging of CRC involving administration of said diagnostic agent to an animate subject and generation of an image of at least part of said subject.

24. A method of generating an optical image of an animate subject involving administering a contrast agent to the subject and generating an optical image of at least a part of the subject to which the contrast agent has distributed, characterized in that a contrast agent as defined in claim 13 is used.
